(19) United States
(12) Patent Application Publication
(10) Pub. No.: US 2003/0220363 A1
(43) Pub. Date: Nov. 27, 2003

(54) Use of propionyl L-carnitine for the preparation of a medicament capable of inducing apoptosis

(75) Inventors: Menotti Calvani, Rome (IT); Claudio Pisano, Aprilia (LT) (IT)

Correspondence Address: NIXON & VANDERHYE, PC, 1100 N GLEBE ROAD, 8TH FLOOR, ARLINGTON, VA, 22201-4714, US

(73) Assignee: SIGMA-TAU INDUSTRIE FARMACEUTICHE RIUNITE S.p.A.

(21) Appl. No.: 10/356,547

(22) Filed: Feb. 3, 2003

Related U.S. Application Data
(62) Division of application No. 09/849,369, filed on May 7, 2001, now abandoned.

(30) Foreign Application Priority Data
Nov. 11, 1998 (IT) RM98A000701
Nov. 11, 1998 (WO) PCT/IT98/00318

(51) Int. Cl. A61K 31/7024; A61K 31/47; A61K 31/198; A61K 31/225

(52) U.S. Cl. 514/300; 514/23; 514/547; 514/563

(57) ABSTRACT

The present invention relates to the use of propionyl L-carnitine and the pharmaceutically acceptable salts thereof for the preparation of medicaments useful in the treatment of pathologies whose treatment gains a benefit from inducement of apoptosis, in particular blood vessels, such as restenosis after angioplasty or coronary stenting, or in particular tumors.

The present invention relates to the use of propionyl L-carnitine and the pharmaceutically acceptable salts thereof for the preparation of medicaments useful in the treatment of pathologies whose treatment gains a benefit from inducement of apoptosis, in particular blood vessels, such as restenosis after angioplasty or coronary stenting, or in particular tumours.

### BACKGROUND OF THE INVENTION

Cell Proliferation in Circulatory Diseases

A number of studies demonstrated that cell proliferation plays a pivotal role in atherosclerosis, hypertension pathogenesis and restenosis after angioplasty or coronary stenting (Ross, 1976; Schwartz, 1990).

Many experimental studies, carried out on human atherosclerotic plaques, demonstrated that cell proliferation is a determining phenomenon both in the early phases and in the progression of the plaque.

Further, proliferation of smooth muscle cells, which migrated to intima from vascular tunica media, represents cell basis of coronaric restenosis—after rivascularization processes through angioplasty or dilatation by means of a stent.

This drawback is the major limit to the application of percutaneous rivascularization in patients affected by acute coronary syndromes, since it is responsible for about 40% of post-surgical failures (Holmes et al., 1984).

Therefore, making available substances capable of controlling the proliferation of smooth muscular cells of vessel wall is a goal of primary importance in the prevention of restenosis after angioplasty, as the proliferative phenomenon occurs in a determined timed corresponding to the first weeks following the intervention.

Proliferation control in experimental atherosclerotic lesions has been obtained with cytostatic drugs, such as etoposide (Llera-Moya et al., 1992), with steroid hormones (Cavallero et al.; 1971; 1973; 1975; 1976), progestinic hormones (Spagnoli et al., 1990), dexamethasone (Asai et al., 1993).

Smooth muscle cell proliferation is also inhibited by calcium antagonist substances due both to a decrease of DNA synthesis, such as in case of verapamil (Stein et al., 1987) and to the interference in second messenger systems (cAMP), as demonstrated for nifedipine (Cheung et al., 1987).

The treatment with ACE-inhibitors resulted in the control of the growth of intima thickening (Powell, 1989).

Other in-vitro studies evidenced an antiproliferative effect on cultured smooth muscular cells of rat aorta given by simvastatine, a HMG-CoA reductase inhibitor, used as hypolipidemic agent (Corsini et al., 1991). Further, some substances having triglyceridhaemia lowering effect, such as fibrates, showed to be able to prevent the progression of atherosclerotic lesions in the human (Olsson et., 1990).

In a manner similar to what observed in neoplasia (Kerr, 1994), phenomena of population decrease are observed to occur together with cell proliferation in atherosclerotic population and/or in intima thickening (Gabbiani, 1995), thus suggesting that highly proliferative cells go toward apoptosis and that modulation of the latter plays an important role in atherosclerotic lesion genesis.

Apoptosis has been demonstrated in various forms of human and experimental cardiovascular diseases (Sharifi AM, Schriffin EL;

Am. J. Hypertens. September 1998; 11(9): 1108-16).

Angioplasty initiates a number of responses in the vessel wall including cellular migration, proliferation, and matrix accumulation, all of which contribute to neointima formation and restenosis (Malik N et al; Circulation Oct. 20, 1998; 98(16): 1657-65). Inducing apoptosis may be beneficial also to reverse vascular disease, as pulmonary vascular disease (Cowan K. N. et al. Circ. Res. 1999 May 28; 84(10): 1223-33). Restenosis after angioplasty is due to damaged intima cells.

Using apoptosis inducing substances, for example for treating tumors, bears the risk to provoke a generalised phenomenon, with possible side effects, which can be even very severe, such as in the case of stem cells.

There is the necessity to find a propaptotic agent devoid of generalised phenomenon.

Proposals for preventing restenosis can be found, for example in U.S. Pat. No. 5,116,864 and U.S. Pat. No. 5,835,935.

Apoptosis in Tumours

It is well-known that the use of anticancer agents in human therapy causes a large number of toxic or side effects which may be life-threatening for the patients. These complications, in fact, may lead to a reduction in the doses of the agents, and occasionally to discontinuation of the therapy itself.

Reduction of the dose or discontinuation of the therapy in many cases causes a deterioration of the individual's general condition because it favours the development of relapses, with consequences which are sometimes fatal for the patient

The growing number and importance of the anticancer agents used in human therapy, the main limitation of which continues to be the occurrence of toxic or side effects, mean that this problem is still a matter for considerable concern.

Thus, the discovery of new agents or new, appropriate combinations of different agents capable of substantially reducing the toxic or side effects caused by anticancer agents used in human therapy is much to be desired.

One of the general problems of pharmacological therapy is the therapeutic index of the agents, that is to say the ratio of the therapeutically effective dose to the toxic dose, or, at any rate, the dose that gives rise to the onset of side effects.

U.S. Pat. No. 5,786,326 a makes a review on the phenomenon of restenosis and indicates that iron is an important requirement for proliferation of SMC. The state of the art therein discussed teaches as a mean for preventing restenosis a drug acting as iron chelating agent. U.S. Pat. No. 5,786,326 provides a new iron chelating agent, namely exochelins.

The medical community still perceives the need for therapeutic regimens which allow the patient to face up to the treatment, which, in the case of anticancer chemotherapy is particularly hard to support, while at the same time conserving an acceptable quality of life. These considerations also apply to the therapeutic treatment of animals, for instance, so-called pets.

The natural tendency to reduce the doses, and thus the use of pharmaceutical forms suitable for therapeutically useful administrations without obliging the patient to take the agents too often, contrasts with the minimum effective doses typical of each anticancer agent.

Thus a substance capable of intervening on the tumour cell, even if devoid of a true cytotoxic activity, but capable of exerting an effect with the antitumour drug, for example inducing apoptosis in the tumour cell, would be of great benefit.

It has now been found that propionyl L-carnitine, thanks to its unexpected proapoptotic effect, is endowed with a specific action of control on smooth muscular cells of vessels and on tumor cells.

Abstract of the Invention.

It is an object of the present invention the use of propionyl L-carnitine and the pharmacologically acceptable salts thereof for the preparation of a medicament useful in the treatment of pathologies whose treatment gains a benefit from inducement of apoptosis, in particular blood vessels, such as restenosis after angioplasty or coronary stenting, or in particular tumors.

WO 97/34596 discloses the use of alkanoyl L-carnitine in the treatment of glutamate mediated diseases, including cancer.

The most important and surprising advantage of the present invention is that the administration of propionyl L-carnitine does not imply toxic effects on bone marrow and in gut, which have a good production of blood cellular elements and a very good turnover of intestinal mucosa cells, respectively. This and other aspects of the present invention will be illustrated in detail, also by means of examples.

Propionyl L-carnitine has been described as preventing the progression of atherosclerotic lesions in aged hyperlipemic rats (Spagnoli L. G., Orlandi A., Marino B., Mauriello A., De Angelis C., Ramacci M. T., Atherosclerosis 1995; 114, 29-44). In this study, the authors demonstrate the strong antiatherogenic effect of propionyl L-carnitine (also named PLC). This effect, although demonstrated in rabbits, admittedly a model not applicable to man, was elucidated through a lipid lowering effect. In fact, the authors state “Although the number of [ . . . ] animals was not very high, it was enough to evidence a strongly significant decrease of total triglycerides, IDL- and VLDL-triglycerides [ . . . ], while the plasma cholesterol level was slightly and transiently modified” (page 40, left-hand column, lines 16-19). The authors also observed a lower level of proliferative activity in both macrophages and SMC composing the plaques (ibid, last 5 lines). The authors then declare that “at present, we cannot assume that PLC has any therapeutic application” (ibid, right-hand column, first 3 lines). However, the results of that study allowed to establish the atherogenic role of β-VLDL in the progression and/or transformation of age-related myointimal thickening in fibroatheromatous plaques (ibid, lines 5-10). The authors provide the hypothesis that plasma triglyceride levels is directly related to the proliferative activity of plaque cell population and that the pharmacological regulation of these two factors may be associated with the marked reduction of the plaque progression in aged hypercholesterolemic rabbits. Also it is stated that some experimental data support the hypothesis of a relationship between triglycerides and cellular proliferation (ibid, page 41, right-had column, lines 20-29). The authors conclude that “PLC does not seem to act through the modulation of the expression of [ . . . ] growth factor” (ibid, last two lines) and that “further in vitro studies are needed in order to answer the question whether or not PLC exerts a direct control on cell proliferation of atherosclerotic plaques” (ibid, page 42, last for lines).

The teaching of this work is that PLC prevents the progression of atherosclerotic lesions in aged hyperlipemic rabbits acting as hypolipidemic agent. Thus, lowering the lipid, which is responsible for cell proliferation of atherosclerotic plaques, PLC indirectly acts as antiatherogenic agent. No antiproliferative action was demonstrated for PLC.

### DETAILED DESCRIPTION OF THE INVENTION.

The present invention is based on the application of the discovery that propionyl L-carnitine (hereinafter also named PLC for brevity) induces the phenomenon of programmed death (apoptosis)

In a later work (Hypertension, Vol 28, No 2, August 1996, pages 177-182), some of the previous authors investigate the effect of PLC on polyploid cells. As explained there, “polyploidy seems to be related to the failure of SMC mitotic division after DNA content duplication”. According to the authors, PLC, although reducing the number of polyploid cells, was found ineffective in controlling blood pressure in SHR. It is also taught that polyploid SMC are hypertension-induced, that the pathophysiological significance of polyploid SMC in SHR aorta is unknown. There follows an explanation of a blockade of the cell cycle. In the hypothesis or the mechanism exerted by PLC there is no suggestion that this compound may have proapoptotic action. in the cells. This effect allows the treatment of blood vessel pathologies based on the proliferation of smooth muscular cells of vessel walls, such as pulmonary hypertension, hypertension, restenosis after angioplasty or coronary stenting.

Advantageously, PLC is a well-known drug, whose side effects are quite limited. Examples of use of propionyl 1-carnitine are U.S. Pat. No. 4,415,589, U.S. Pat. No. 4,255,449, IT 1155772, EP 0793962, EP 0811376, WO99/17623, PCT/IT97/00113.

Accordingly, a first aspect of the present invention relates to the use of propionyl L-carnitine and the pharmacologically acceptable salts thereof for the preparation of a medicament useful in the treatment of pathologies whose treatment gains a benefit from inducement of apoptosis, in particular blood vessels, such as restenosis after angioplasty or coronary stenting, or in particular tumors.

Another object of the present invention is the use of propionyl L-carnitine and the pharmacologically acceptable salts thereof for the preparation of a medicament useful for the treatment of hypertension.

Another object of the present invention is the use of propionyl L-carnitine and the pharmacologically acceptable salts thereof for the preparation of a medicament useful for the treatment of pulmonary hypertension.

Still another aspect of the present invention is the use of propionyl L-carnitine and the pharmacologically acceptable salts thereof for the preparation of a medicament useful to prevent restenosis after angioplasty or coronary stenting.

Also an object of the invention described herein is the co-ordinated use the propionyl L-carnitine according to which an adjuvant effect with the anticancer agent is obtained. As adjuvant effect it is intended a combination therapy of an antitumour drug and propionyl L-carnitine, by means of which, PLC exerts an apoptotic effect on the tumour cell, thus assisting the cytotoxic effect of the antitumour drug. In this way an improvement of the therapeutic index of the antitumour drug is expected.

A further object of the invention described herein is the use of propionyl L-carnitine in the preparation of a medicament useful for treating tumors.

Yet another object of the invention described herein are combinations the propionyl L-carnitine with anticancer agents and the related pharmaceutical compositions.

In the context of the invention described herein, what is meant by “co-ordinated use” of the aforesaid compounds is, indifferently, either (i) co-administration, i.e. the substantially simultaneous or sequential administration of propionyl L-carnitine or one of its pharmacologically acceptable salts and of an anticancer agent, or (ii) the administration of a composition comprising the aforesaid active ingredients in combination and in a mixture, in addition to optional pharmaceutically acceptable excipients and/or vehicles.

The invention described herein thus covers both the co-administration of propionyl L-carnitine or one of its pharmacologically acceptable salts and of the anticancer agent, and pharmaceutical compositions, which can be administered orally, parenterally or nasally, including controlled-release forms, comprising the two active ingredients in a mixture.

Though clear from the following detailed description of the invention, one can also envisage the coordinated use of an anticancer agent, such as for example, taxol, bleomycin, carboplatin, vincristine, a camptothecine. In all these embodiments, propionyl L-carnitine can be used in the co-ordinated use.

Co-administration also means a package, or manufactured article, comprising distinct administration forms of propionyl L-carnitine or one of its pharmacologically acceptable salts and of an anticancer agent, accompanied by instructions for the co-ordinated simultaneous or time-scheduled intake of the active ingredients according to a dosage regimen established by the primary care physician, on the basis of the patient's condition.

The embodiment of the invention described herein also contributes to healing and to prolonging the lives of the patients thanks to the increase in therapeutic success due to the possibility of maintaining the scheduled treatment protocols or of increasing the doses of the chemotherapeutic agent, without having to discontinue the treatment due to contraindications. It also possible to foresee a reduction of the dose of the anticancer drug, thanks to the adjuvant effect of propionyl L-carnitine.

The medicament according to the present invention can be obtained admixing the active ingredient (propionyl L-carnitine or a pharmacologically acceptable salt thereof) with excipients suitable for formulation of compositions intended for enteral administration (in particular the oral one) or parenteral administration (in particular through intramuscular or intravenous route). All such excipients are well known to persons skilled in the art.

As pharmaceutically acceptable salt of propionyl L-carnitine, it is intended any salt thereof with an acid which does not give rise to unwanted side effects. These acids are well known to the pharmacologists and to the experts of pharmaceutical technology.

Non-limiting examples of said salts are chloride, bromide, orotate, acid aspartate, acid citrate, acid phosphate, fumarate and acid fumarate, lactate, maleate and acid maleate, acid oxalate, acid sulphate, glucose phosphate, tartrate and acid tartrate.

Some examples of formulations in the form of unitary dosages are given.

(a) Formulation for tablets

| A tablet contains: | | |
|---|---|---|
| Active ingredient | | |
| propionyl L-carnitine HCl | mg | 500 |
| Excipients | | |
| Microcrystalline cellulose | mg | 54.0 |
| Polyvinylpyrrolydone | mg | 18.0 |
| Crospovidone | mg | 30.0 |
| Magnesium Stearate | mg | 15.0 |
| Fumed silica | mg | 3.0 |
| Hydroxypropylmethylcellulose | mg | 10.0 |
| Poliethylene glycole 6000 | mg | 2.5 |
| Titanium dioxide | mg | 1.8 |
| Methacrylate copolymer | mg | 8.3 |
| Talcum (triventilated) | mg | 2.4 |

(b) Formulation of intravenously injectable bottles

| A bottle contains: | | |
|---|---|---|
| Active ingredient | | |
| Propionyl L-carnitine HCl | mg | 300 |
| Excipient | | |
| Mannitol | mg | 300 |
| A solvent vial contains: | | |
| Sodium acetate 3.H₂O | mg | 390 |
| Water for injectable F.U. q. s. to | ml | 5 |

The medicament prepared according to the present invention will be administered in the form of pharmaceutical composition, which can be prepared according to the general common knowledge of the person skilled in the art.

Depending on the administration route appropriately chosen, oral, parenteral or intravenous; the pharmaceutical composition will be in the suitable form.

Examples of pharmaceutical compositions, wherein the medicament according to the present invention is comprised, are the solid or liquid oral forms, such as tablets, all types of capsules, pills, solutions, suspensions, emulsions in the form of unitary or divided doses, syrups, ready-to-use or extemporary drinkable unit doses. Other examples are parenteral forms, injectable forms for intramuscular, subcutaneous or intravenous administration. Controlled or programmed release forms are also appropriate.

Dosages, posology and general therapeutic regimen will be determined by the physician according to his knowledge, patient's conditions and the pathology to be treated.

The association, whether co-administered in the same medicament or separately (at the same time or subsequently) of PLC with other active ingredients is also comprised in the present invention.

In a first preferred embodiment, the present invention relates to restenosis after angioplasty.

According to this first preferred embodiment, the pharmacological dose of PLC is such as not to exceed hematic concentration of 100 mM.

The following example further illustrate the invention.

### EXAMPLE 1

Wistar male rats, weighing between 270 and 290 mg, were used for the experiments. The rats were anaesthetised with Nembutal i.p. (35 mg/kg body weight) and the thoracic portion of aorta was submitted to endothelium mechanical removal with Fogarty 2F balloon probe (Baxter USA), according to the Baugartner and Studer method(1966) with minor modification (Orlandi 1994). The animals were randomized into 5 groups, each group is reported in Table 1.

Two groups were subjected to pharmacological treatment with propionyl L-carnitine (PLC, 120 mg/Kg p.c. die), one group was treated with an ACE-inhibitor (Enalapril, 1 mg/Kg p.c. die); the two remaining groups were the control. Moreover, some non-balloonized animals were used as blanks.

TABLE 1

| Final number | | Duration |
|---|---|---|
| of Wistar rats | Treatment | (days) |
| 7 | de-endothelialization + PLC | 3 |
| 7 | de-endothelialization | 3 |
| 8 | de-endothelialization + ACE-antagonist | 15 |
| 8 | de-endothelialization + PLC | 15 |
| 8 | de-endothelialization | 15 |
| 5 | blanks | — |

The animals were sacrificed 3 and 15 days after de-endothelialization. Two hours before sacrifice, all the rats received i.v. a Bromodeoxyuridine solution (BrDU) (45 mg/kg body weight) in order to verify cell proliferation. One hour before sacrifice, some randomly selected animals received 1 ml Blue Evans (1% in 0.9% NaCl solution) in order to evaluate the degree of aorta disruption.

At sacrifice, the animals were anaesthetised with i.p. Nembutal and perfused, after washing with isotonic saline containing 3% Dextran 70, with buffered formalin for 20 minutes. Aortae were isolated, slightly washed in saline and dissected longitudinally. Carotid, heart and small intestine were also excised. All the organs were post-fixed in the same fixative for 24 hours at room temperature.

Some aortic fragments were used for electronic microscopy. Aortae were rolled up and included in paraffin. Serial sections having 5 μm thickness were stained with Hematoxylin-Eosine, Verhoeff-Van Gieson and Movat's pentachromic and used for morphologic and morphometric studies.

In some non-perfused animals, fragments of aortic tissue were frozen in liquid nitrogen for the determination of tissular carnitines and for subsequent studies of molecular biology.

Immunohystochemical Staining

In order to put in evidence proliferating cells in damaged arteries, serial sections in paraffin of aortae were deparaffined, rehydrated, immersed in a 3% H₂O₂ solution for 20 minutes and incubated with trypsine (0.05 M in Tris-HCl, pH 7.6) at 37° C. After that, sections were treated with 2N HCl at 37° C. for 30 minutes, washed with 0.1 M sodium tetraborate for 10 minutes, incubated with normal horse serum (Vector) and subsequently with and-BrDU monoclonal antibody (Ylem) for 1 hour. The preparates were then reacted with biotilinated anti-mouse IgG (Vector) and the Streptoavidine-ABC-POD complex (Ylem).

The reaction was evidenced by using diaminobenzidine (DAB) as final chromogen. The count of positive nuclei for BrDU was made on the total number of nuclei. Such count was blind-made by two researchers separately. The difference between the two counts was always lower than 0.5%.

All data were analysed with the T Student's test. The differences between the groups were considered to be significant for P<0.05.

Morphometric Analysis

The entity of intima thickening after 15 days was evaluated on Verhoeff-Van Gieson stained sections, using a grid overlapped on the image, consisting of 400 points, 1 cm from each other.

The analysis was made on hystological preparates through a Hamamatsu C3077 camera controlled by a Hamamatsu DVS 3000 image analyser and connected to a Polyvar-Reichert microscope. Morphometric evaluation was made at X116 magnification. The following parameters were evaluated a) relative volume of intima referred to arterial wall; b) relative volume of tunica media referred to arterial wall, by counting the overlapping points on the intima and mean tunica.

3-12 aorta sections were used at different level for each animal. This number was a function of the structure sizes, according to Sach's formula, showing the number of fields necessary to obtain a statistically significant sample.

Ultrastructural Studies

Small aorta samples were selected for electronic microscopy. Aortae were post-fixed in osmium tetraoxide and embedded in EPON 812. Ultra thin sections were stained with uranyl acetate followed by lead citrate and examined using a Hitachi H-7100 FA transmission electronic microscope.

Tissular and Plasma Carnitine Assay

2-3 ml of blood samples were withdrawn from each animal before mechanical de-endothelialization and at the time of sacrifice. Plasma was separated by centrifugation (300 rpm) for 20 minutes and frozen for plasmatic carnitine assay according to the Pace et al. method.

Aorta wall samples were withdrawn from some non-perfused animals, randomly selected from each group, frozen in liquid nitrogen and stored at −80° C. for the carnitine assay, according to the above Pace et al. reference.

Results

Ultrastructural Studies

Small aorta samples were selected for electronic microscopy. Aortae were post-fixed in osmium tetraoxide and embedded into EPOC 812. Ultra thin sections were stained with uranyl acetate followed by lead citrate and examined through a Hitatchi H-7100 FA transmission electron microscope.

Tissular and plasmatic carnitine assay 2-3 ml blood samples were withdrawn from each animal before mechanical de-endothelialization and just before sacrifice. Plasma was separated by centrifugation (3000 rpm) for 20 minutes and frozen for the plasmatic carnitine assay according to the Pace et al. method.

Samples of aortic wall were taken from some non-perfused animals, randomly selected from each group, frozen in liquid nitrogen and kept at −80° C. for the assay of tissutal carnitines according to the above-mentioned Pace et al. method.

Results

Lesion Morphology

3 days after the mechanical lesion, rat aortae did not show significant hystological alterations, except the lack of endothelial cell coating.

15 days after, remodelling of arteria could be observed for the presence of an intima thickening (or neointima), consisting in round or lengthened cells immersed in abundant extracellular matrix. Immune hystochemical study put in evidence in particular the presence of abundant smooth muscular cells (SMC) inside neointima.

Studies on Proliferation

a) 3 days after de-endothelialization: the count of anti-BrDU staining positive nuclei showed substantial differences between the two groups examined. Quantitative analysis (Table 2) puts in evidence that the number of BrDU-positive nuclei is significantly lower in the tunica media in the PLC-treated animals, with respect to controls (59.3% reduction against control, p<0.02). In both groups the distribution of BrDU-positive nuclei is more concentrated in the lumen portion of mean tunica with respect to the adventitia portion, with a 2:1 ratio.

b) 15 days after de-endothelialization: Table 3 shows that in each group the proliferation index of SMCs is significantly higher (p<0.001) in the intima with respect to the tunica media. No significant differences are observed in the number of BrDU-positive nuclei, in the intima and tunica media, by comparing PLC, Enalapril and control animals.

Morphometric Analysis

As described in Table 3, after 15 days from endothelial lesion, the intima relative volume is significantly lower, both in the PLC-treated (31.11% reduction against control, p<0.02) and ACE-antagonist-treated (26.14% reduction against control, p<0.01) animals against control animals.

TABLE 2
In-vivo treatment with propionyl L-carnitine (PLC) on the
proliferation of smooth muscle cells of rat aorta after mechanical
de-endothelialization: percentage of proliferating cell nuclei (anti-
bromodeoxyuridine positive) after 3 days (± s.e.m)

| | | positive nuclei/ | | |
|---|---|---|---|---|
| | interval | total nuclei % | Reduction % | Difference |
| Mean tunica | 3 days | 6.36 + 1.27 | | |
| Control animals | | | | |
| Tunica media PLC | 3 days | 2.59 ± 0.56 | 59.3 | P < 0.02 |
| treated animals | | | | |

TABLE 3
In vivo treatment with propionyl L-carnitine (PLC) and with the
ACE-antagonist Enalapril on the proliferation of smooth muscular
cells of rat aorta after mechanical de-endothelialization: percentage
of proliferating cells (anti-bromodeoxyuridine positive) and
percentage ratio between intima volume and aorta wall volume after
15 days (± s.e.m.) (preliminary results).

| | | +Nuclei/ | Intima | Reduction |
|---|---|---|---|---|
| | interval | total nuclei % | volume/wall | (% to CTR) |
| Intima tunica, | 15 days | 2.65 ± 0.44 (a) | 29.73 ± 1.54 | |
| control animals | | | | |
| Intima tunica, | 15 days. | 1.99 + 0.32 (b) | 20.48 ± 2.73 ^{(d)} | 31.11 |
| PLC treated | | | | |
| animals | | | | |
| Intima tunica, | 15 days | 2.43 ± 0.39 (c) | 21.96 ± 1.20 ^{(e)} | 26.14 |
| enalapril | | | | |
| treated animals | | | | |
| Tunica media, | 15 days | 0.24 ± 0.05 | | |
| control animals | | | | |
| Tunica media, | 15 days | 0.24 ± 0.09 | | |
| PLC treated | | | | |
| animals | | | | |
| Tunica media, | 15 days | 0.27 ± 0.09 | | |
| enalapril | | | | |
| treated animals | | | | |
| Tunica media, | | 0.24 ± 0.05 | | |
| non peeled | | | | |
| animals | | | | |

(a) intima vs tunica media: p < 0.0001;
(b) intima vs tunica media: p < 0.0001;
(c) intima vs tunica media: p < 0.001;
^{(d)} intima vol./wall vs controls: p < 0.02;
^{(e)} intima vol/wall vs controls: p < 0.01

Effect of Propionyl L-Carnitine in the Control of Proliferation/Apoptosis.

In vitro experiments were carried out to evaluate the effect of propionyl L-carnitine (PLC) on smooth muscular cells (SMC) isolated from aortae of spontaneously hypertensive rats (SHR) and, as control, on SMC isolated from normotensive rats (WKY).

These in vitro studies evidenced that PLC, when administered during culture exponential growth phase, reduces cell growth, evaluated as cell number/ml, as well as DNA synthesis, evaluated through incorporation of trititated thymidine (Tab. 4 and 5).

TABLE 4
cell number/ml at culture days 2, 3, 4 and 6

| | Day 2 | Day 3 | Day 4 | Day 6 |
|---|---|---|---|---|
| SHR CTRL | 5*10⁴ ± 2 | 15*10⁴ ± 3 | 28*10⁴ ± 7 | 42*10⁴ ± 7 |
| SHR PLC | 7*10⁴ ± 4 | 4*10⁴ ± 2 | 7*10⁴ ± 4 | 20*10⁴ ± 5 |
| WKY CTRL | 4*10⁴ ± 1 | 2*10⁴ ± 2 | 7*10⁴ ± 2 | 13 − 10⁴ ± 4 |
| WKY PLC | 3*10⁴ ± 2 | 4*10⁴ ± 2 | 6*10⁴ ± 2 | 8*10⁴ ± 3 |

TABLE 5
tritiated thymidine incorporation at culture day 6

| | μCi/μgDNA |
|---|---|
| SHR CTRL | 2.99E−05 |
| SHR PLC | 1.81E−05 |
| WKY CTRL | 2.1E−05 |
| WKY PLC | 2.56E−05 |

As a further characterisation of smooth muscular cells in the presence of PLC, the percentage of apoptotic cells was measured both in basal conditions and in oxidative stress conditions. Apoptosis evaluation was carried out by counting the number of apoptotic cells present on a total of 1000 cells, after specific DNA staining with Hoechst 33258. The results of this experiment demonstrated that, in SHR cultures, PLC determines a significant increase of apoptosis percentage in basal conditions and that this increase is more evident under stress conditions.

In WKY cultures, apoptosis percentage is negligible (tab. 6)

TABLE 6
apoptotic cell percentage in basal conditions and under oxidative stress.

| | Basal conditions | Oxidative stress |
|---|---|---|
| SHR CTRL | 0 | 2 |
| SHR PLC | 2 | 10 |
| WKY CTRL | 0 | 0 |
| WKY PLC 2 | 0 | 2 |

The behaviour observed in SHR smooth muscular cells might be in some way related to the deregulated expression of c-myc, which characterises spontaneously hypertensive rats (Negoro et al., 1988). Moreover, it was observed that c-myc actively cooperates in inducing apoptosis subsequently to a proliferation stop (Bennet et al., 1993; Bissonette et al., 1993), accordingly the data shown above suggest that PLC anti-proliferative effect may be related to an interference with DNA replication.

### EXAMPLE 2

Cell Lines

Human derived neoplastic cell, obtained from Istituto Zooprofilattico of Brescia, were cultivated. The cells used for the experiment were: U266, multiple myleopma, HeLa, uterine cervix tumor, K562, chronic myeloid leukemia cells. HeLa and K562 were cultivated in RPMI+10% FCS, while those of U266 line were cultivated in RPMI+15% FCS, both media containing Penicilline/Streptomycine (50 U/mL and 50 μg/mL). Cells were plated in 6-wells plates (Falcon). Analysis were performed in cells having 50% confluence.

Each cell line was treated with PCL according to the folowing schemes:

a) 1 mM PLC for 24 hours (FIGS. 1-3); 1 mM PLC for 48 hours (FIGS. 1-3);

b) 1 mM PLC for 24 hours, followed by 24 hours media without molecules (FIGS. 4-6).

At the end of each treatment, cells were counted in Burker chamber in the presence of Trypan Blue 0.5%, diluted 1:2. Count was made for each experimental group on samples coming from three wells.

The treatment of neoplastic cell lines of human origin with 1 mM propionyl L-carnitine show the capacity of inhibiting the proliferation both after 24 hours and after 48 hours. In particular inhibition was respectively 25% and 17% after 24 and 48 hours with respect to the control for HeLa; 46% and 26% after 24 and 48 hours with respect to the control for U266; 37% and 39% after 24 and 48 hours with respect to the control for K562.

PLC inhibition effect persists after having removed the substances from cultivation medium. In fact, in this case cells werer treated for 24 hours with 1 nM PLC, then the medium contining PLC was removed and fresh medium was added without the substance. Inhibition values were 20%, 26% and 21% for HeLa, U266 and K562, respectively.

### REFERENCES

Spagnoli L. G. Giorn. Arterioscl. 1983; 8: 117-145

Asai K, Funaki C, Hayashi T. et al. Arterioscl. Thromb. 1993; 13: 892-899

Baumgartner H. R., Studer A, Pathol. Microbiol. (Base) 29: 393-405, 1966

Bennet M. R., Evan G. I., Newby A. C., Circ. Res. 1994; 74:525-536

Bissonette R. P., Shi Y., Mahboubi A., Glynn J. M. and Green D. R, Curr. Commun. Cell. Mol. Biol. 1993

Bonanno E., Ghibelli L., Coppola S., Spagnoli L. G., International Conference on Cell Death in Human Pathology. Lecce, Jun. 22-25, p. 54, 1995

Bouchaton-Piallat M. L., Gabbiani F., Desmouliere A. and Gabbiani G. Am. J. Pathol. 146: 1059-1064, 1995

Cavallero C., De Lellis C., Di Tondo U. et al. In: Cavallero C., editor. The arterial wall in atherogenesi. Padova: Piccin Medical Book, 1975: 25-42

Cavallero C., Di Tondo U. Mingazini L. et al., Aherosclerosis 1973; 17: 49-62

Cavallero C., Di Tondo U., Mingazzini P. et al. Atherosclerosis 1976; 25: 145-152

Cavallero C., Turolla E. and Ricevuti G., Atherosclerosis 1971; 13: 9-20

Cheung W. T., Shi M. N., Young J. D. et al., Biochem. Pharmacol. 1987; 36: 2183-2189

Clowes A. W., Schwartz S. M., Cir. Res. 56: 139-145, 1985

Corsini A., Raiteri M., Soma M. et al., Pharmacol. Res. 1991; 23: 173-180

Curi R., Bond J. A., Calder P.C. and Newsholme E. A., Gen. Pharmac. 1993; 24, 591-597

Holmes D. R. J., Vliestra R. E., Smith H. C., Am. J. Cardiol. 1984; 77C-81C.

Kerr J. F. R., Winterford C. M., Harmon B. V., Cancer 73: 2013-2026, 1994

Llera-Moya M., Rothblat G.H., Glick J.M. et al., Arterioscler. Thromb. 1992; 12: 1363-1370

Mauriello A., Sangiorgi G., Orlandi A., Schiaroli S., Pertumo S., Spagnoli L. G.,

Negoro N., Inariba H., Inoue T., Kanayama Y., Takeda T.

Olsson A.G., Ruhn G., Erikson U., J. Int. Med. 227: 381, 1990

Orlandi A., Ehrlich H. P., Ropraz P. et al., Arterioscler. Thromb. 1994; 14: 982-989

Orlandi A., Ropraz P. and Gabbiani G., Exp Cell Res 1994; 214: 528-536

Powell J. S., Clozel J-P., Muller R. K. M., Kuhn H, Hefti F., Hosang M., Baumgarner H., Science 245: 186-188, 1989

Ross R., Glomset J. A., N. Engi. J. Med. 295: 369-377, 1976

Schwartz S. M., Heimark R. L., Majesky M. V., Physiol. Rev. 70: 1177-1209, 1990

Spagnoli L. G., Giorn. Arterioscl. 1983; 8: 117-145

Spagnoli L. G., Orlandi A., Marino B., Mauriello A., De Angelis C., Ramacci M. T., Atherosclerosis 1995; 114, 29-44

Spagnoli L. G., Orlandi A., Marino B. et al., Atherosclerosis 1995; 114: 29-44

Spagnoli L. G., Orlandi A., Mauriello A., et al., Pathol. Res. Pract. 1992; 4-5: 637642

Spagnoli L. G., Orlandi A., Mauriello A. et al. Atherosclerosis 1991; 89: 11-24.

Spagnoli L. G., Palmieri G., Mauriello A. et al., Atherosclerosis 1990; 82: 27-36

Spagnoli L. G., Sarnbuy Y., Palmieri G. et al., Artery 1985; 13: 187-198

Stein O., Halperin G., Stein Y., Arteriosclerosis 1987; 7: 585-592

1. Use of propionyl L-carnitine or a pharmaceutically acceptable salts thereof for the preparation of a medicament useful in the treatment of pathologies whose treatment gins a benefit from inducement of apoptosis.

2. Use according to claim 1, wherein said medicament is useful for the treatment of hypertension.

3. Use according to claim 1, wherein said medicament is useful for the treatment of pulmonary hypertension.

4. Use according to claim 1, wherein said medicament is useful for the prevention of restenosis after angioplasty or coronary stenting.

5. Use according to claim 1, wherein said medicament is useful for the treatment of tumors.

6. Use according to anyone of claims 1 to 5, wherein said salt of propionyl L-carnitine is selected from the group consisting of chloride, bromide, orotate, acid aspartate, acid citrate, acid phosphate, fumarate and acid fumarate, lactate, maleate and acid maleate, acid oxalate, acid sulphate, glucose phosphate, tartrate and acid tartrate.

7. Combination comprising an antitumour drug and propionyl L-carnitine, with the proviso that said antitumour drug is not doxorubicin.

8. Use of the combination according to claim 7 for the preparation of a medicament with anticancer activity, characterised in that said medicament comprises an effective amount of propionyl L-carnitine which exerts an adjuvant action for the anticancer activity.

9. A package, or manufactured article, comprising distinct administration forms of propionyl L-carnitine, or one of its pharmacologically acceptable salts and of an anticancer agent, accompanied by instructions for the coordinated simultaneous or time-scheduled intake of the active ingredients, with the proviso that said antitumour drug is not doxorubicin.
